(19) United States
(12) Patent Application Publication
(10) Pub. No.: US 2023/0404399 A1
(43) Pub. Date: Dec. 21, 2023

(54) ADAPTOR AND METHOD FOR CAPTURING IMAGES OF A RETINA OF AN EYE

(71) Applicant: C-MER RAINSOPTICS LIMITED, HONG KONG, HONG KONG (CN)

(72) Inventor: Ho Wa LAI, Hong Kong (CN)

(73) Assignee: C-MER RAINSOPTICS LIMITED, HONG KONG HONG KONG (CN)

(21) Appl. No.: 18/251,363

(22) PCT Filed: Aug. 4, 2021
(86) PCT No.: PCT/IB2021/057129

§ 371 Date: May 1, 2023

Related U.S. Application Data
(60) Provisional application No. 63/108,524, filed on Nov. 2, 2020.

(51) Int. Cl. A61B 3/14; A61B 3/00; A61B 3/12

(52) U.S. Cl. CPC A61B 3/14; A61B 3/0008; A61B 3/1208

(57) ABSTRACT

An adaptor (100) which may be attached to a portable image capturing device (19) and a method of capturing images of a retina (2) of an eye (1). The adaptor (100) may comprises an illumination unit (110), a beam splitter (130), an objective lens system (140) and a secondary lens system (150). The objective lens system (140) and the secondary lens system (150) share a first optical axis (160). By offsetting an output aperture (108) of the adaptor (100), or offsetting a camera of the image capturing device (19), from the first optical axis (160) and/or offsetting the illumination unit (110) from a second optical axis (170), overlap of imaging and illumination pathways in the eye (1) may be reduced or eliminated.

### CROSS-REFERENCE TO RELATED APPLICATIONS

This application is a national phase application of International Application No PCT/IB2021/057129, filed Aug. 4, 2021, which claims priority to U.S. Patent Application Ser. No. 63/108,524, filed Nov. 2, 2020, all of which are incorporated herein by reference.

### BACKGROUND

The present disclosure relates to an adaptor for attachment to a portable image capturing device for capturing images of the retina of an eye and a method of capturing images of the retina of an eye.

FIG. 1 shows the basic structure of a human eye 1 which comprises a cornea 5, a pupil 4, a lens 3, and a retina 2. The retina 2 is an internal light sensitive layer at the back of the eye 1 and is primarily responsible for vision. Light from a distant object or light source 10 travelling in a near parallel path enters the eye through the pupil 4 and is focused at a point on the retina 2 by the refractive power of the cornea 5 and lens 3. The light focused on the retina 2 is detected by photoreceptor cells of the retina 2, and converted into an electrical signal. The electrical signal is transferred by retinal ganglion cells, through the optic nerve of the eye 1, into the brain for visual processing. This is generally how a human can see the outside world.

Unlike the cornea 5, the retina 2 is currently not replaceable. Currently there is no artificial retina or other substitutes that can provide sufficient visual function in the event the retina fails. Unfortunately, the retina 2 is quite vulnerable to various problems and diseases and is therefore subject to failure. Consequently, care should be taken to ensure the health of the retina. Furthermore, since the retina is the only portion of the central nervous system visible from outside the human body, inspection of the retina can enable detection of other health issues such as diabetes. Therefore, examination of the retina 2 is one of the most important aspects of an eye examination because it enables the detection and prevention of pathological conditions that can result in irreversible visual loss or other health related issues.

An eye examination is traditionally carried out by a specialist eye doctor, commonly referred to as an ophthalmologist, who visually inspects the fundus of the eye using an ophthalmoscope. One limitation of an ophthalmoscope is that it is unable to contemporaneously record visual details of the fundus which means that the ophthalmologist is required to subsequently document his findings of the visual inspection of the retina in text or drawings. Accurate recording or documentation of images of the fundus require another instrument commonly referred to as a retinal camera or fundus camera. The process of taking photographs of the retina is called fundus photography. Fundus photography provides photographic documentation of the retina and facilitates documentation, monitoring, case discussion, mass screening, and even telemedicine.

Conventional fundus cameras are usually large machines that must be table mounted and connected to a desktop computer system for image storage and organisation. Such conventional cameras are not helpful for bed-bound patients, infants and children, or other patients that cannot easily move or cooperate for accurate positioning relative to the camera. Furthermore, such cameras limit the examination to the clinic or hospital. Outreach screening with such fundus cameras is therefore very difficult.

Recently, a number of portable fundus cameras have been developed to address these mobility issues. These portable fundus cameras have greatly expanded the ability to conduct funduscopic or ophthalmoscopic examinations. However, portable fundus cameras still require relatively complicated connection to a computer system for photo storage, processing and organisation. Auto-analysis and telemedicine is possible with such cameras, but is still limited to specialist centres that have the dedicated facilities and computer systems for assessment.

With the advent of smartphones and other portable image capturing devices, retinal imaging with smartphones is gaining popularity. One advantage of using a smartphone for retinal imaging is that it does not require connection to remote computer systems. Smartphones also allow instant image capture, review, analysis, organisation and sharing of fundus photographs. However, due to limited field of view, smartphones produce poor quality images of the retina when used alone. Furthermore, fundus photography is usually performed on eyes whose pupils have been dilated and are thus in a mydiatric state. This requires use of eye-dilating medicine, such as eye drops, administered by a qualified clinician. It would be desirable to perform findus photography on eyes in a non-dilated (non-mydiatric) state, but due to the low pupil size, it is difficult to achieve sufficient illumination of the retina, when the eye is in a non-mydiatric state.

### SUMMARY

The present disclosure proposes an adaptor which may be attached to a portable image capturing device, such as but not limited to a smart phone, for capturing images of a retina, also referred to in this disclosure as fundus photography.

A first aspect of the present disclosure provides an adaptor comprising an illumination unit, a condensing lens system, a beam splitter, an objective lens system, a secondary lens system and an output aperture which is to be positioned adjacent a camera of the portable image capturing device. The illumination unit is configured to direct the optical radiation on an illumination path from the illumination unit through the condensing lens system to the beam splitter. The beam splitter is positioned between the objective lens system and the secondary lens system and is configured to direct the optical radiation on the illumination path to the objective lens system for illuminating the retina. The objective lens system is configured to focus the optical radiation on the illumination path onto a focal plane positioned between a cornea and a backside of a crystalline lens of the eye and direct optical radiation reflected from the retina on an imaging path to the secondary lens system. The secondary lens system is configured to direct the optical radiation on the imaging path through the output aperture. The objective lens system and the secondary lens system share a first optical axis. The condensing lens system has a second optical axis which is at an angle to the first optical axis. The illumination unit is offset from the second optical axis and/or a centre of the output aperture of the adaptor is offset from the first optical axis.

A second aspect of the present disclosure provides an adaptor comprising an illumination unit, a beam splitter, an objective lens system and a secondary lens system. The illumination unit comprises an optical radiation source and is configured to direct optical radiation from the optical radiation source to the beam splitter. The beam splitter is positioned between the objective lens system and the secondary lens system and is configured to direct the optical radiation to the objective lens system for illuminating the retina. The objective lens system is configured to focus the optical radiation on a focal plane positioned between a cornea and a backside of a crystalline lens of the eye and to direct optical radiation reflected from the retina towards the secondary lens system. The secondary lens system is configured to direct the reflected optical radiation to an exterior of the adaptor for reception by a camera of a portable image capturing device which is to be attached to the adaptor. The objective lens system and the secondary lens system share a first optical axis and the adaptor is configured to attach to the portable image capturing device in a position in which the camera of the portable image capturing device is offset from the first optical axis.

A third aspect of the present disclosure provides a method of capturing an image of a retina of an eye. The method comprises attaching an adaptor to a portable image capturing device including a camera; generating optical radiation by an optical radiation source of an illumination unit of the adaptor; directing the optical radiation from the illumination unit through a condensing lens system on an illumination path to a beam splitter and from the beam splitter to an objective lens system; focusing the illumination path optical radiation by the objective lens system onto a focal plane positioned between a cornea and a backside of a crystalline lens of the eye, wherein the eye is not in contact with the adaptor; receiving, by the objective lens system, optical radiation reflected by the retina of the eye and directing the reflected optical radiation on an imaging path through the beam splitter to a secondary lens system and directing optical radiation on the imaging path, by the secondary lens system, onto a camera of portable image capturing device. The objective lens system and the secondary lens system share a first optical axis and the condensing lens system has a second optical axis which is at an angle to the first optical axis. The camera of the portable image capturing device is offset from the first optical axis and/or an illumination aperture associated with the illumination unit is offset from the second optical axis.

### BRIEF DESCRIPTION OF THE DRAWINGS

Examples of the present disclosure will be explained below with reference to the accompanying drawings, in which:—

FIG. 1 is a schematic diagram showing features of an eye;

FIG. 2A is a schematic diagram showing an adaptor according to an example of the present disclosure;

FIG. 2B is a schematic diagram showing another adaptor according to an example of the present disclosure;

FIG. 3 is a schematic diagram showing an illumination path in an adaptor according to an example of the present disclosure;

FIG. 4 is a schematic diagram showing an imaging path in an adaptor according to an example of the present disclosure;

FIG. 5 is a schematic diagram showing a field of view of an imaging path in an adaptor according to an example of the present disclosure;

FIG. 6 is a schematic diagram showing both an illumination path and an illumination path in an adaptor according to an example of the present disclosure;

FIG. 7 is a schematic diagram showing an illumination path and an imaging path of optical radiation in an eye from an adaptor according to an example of the present disclosure;

FIG. 8 is a schematic diagram showing real images formed on a focal plane of an eye by light from an imaging path and an illumination path of an adaptor according to an example of the present disclosure;

FIG. 9 is schematic diagram showing the polarization of light in an adaptor according to an example of the present disclosure;

FIG. 10 is a schematic diagram showing an adaptor according to an example of the present disclosure when in use and attached to a image capturing device and placed adjacent an eye whose retina is to be photographed;

FIG. 11 is a schematic diagram showing an adaptor according to an example of the present disclosure;

FIG. 12 is a schematic diagram showing a positional relationship of a mount, camera of an imaging capturing device and secondary lens system of an adaptor according to an example of the present disclosure; and

FIG. 13 is a flow diagram illustrating a method of capturing images of a retina of an eye according to an example of the present disclosure.

### DETAILED DESCRIPTION

Various examples of the disclosure are discussed below. While specific implementations are discussed, it should be understood that this is done for illustrative purposes and variations with other components and configurations may be used without departing from the scope of the disclosure as defined by appended claims.

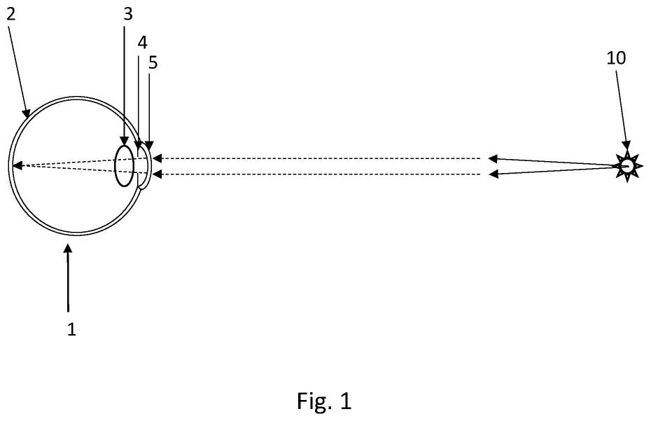

Referring to FIG. 1, the basic principle of fundus photography is for a camera to capture light rays reflected from the retina 2. In order to capture an image having sufficient quality for medical analysis, it is generally necessary to illuminate the retina with a source of optical radiation 10, such as a lamp or light emitting diode (LED). In the context of this disclosure optical radiation means electromagnetic radiation in the visible or infrared spectrum and references to light are to be interpreted to include electromagnetic radiation having a wavelength in the visible or infrared spectrum.

In fundus photography, light from the source of optical radiation travels into the eye through the pupil 4 and light reflected by the retina exits the eye through the pupil 4. The reflected light (not shown in FIG. 1) can then be captured by a camera (not shown in FIG. 1). However light incident the eye from the light source may interfere with the light reflected from the retina, resulting in a poor quality image. One solution to this is to use a ring shaped light source which forms an annular image on the pupil plane of the eye and for the camera to capture light reflected from the retina through the annular ring of illuminating light in the pupil plane. In this way light on the illumination path incident the eye does not interfere with light on the imaging path reflected from the retina. However, this approach requires a relatively large pupil in order that the ring of illuminating light can pass through the pupil. Accordingly, this approach is not suitable for non-mydiatric fundus photography, in which the eye is not dilated.

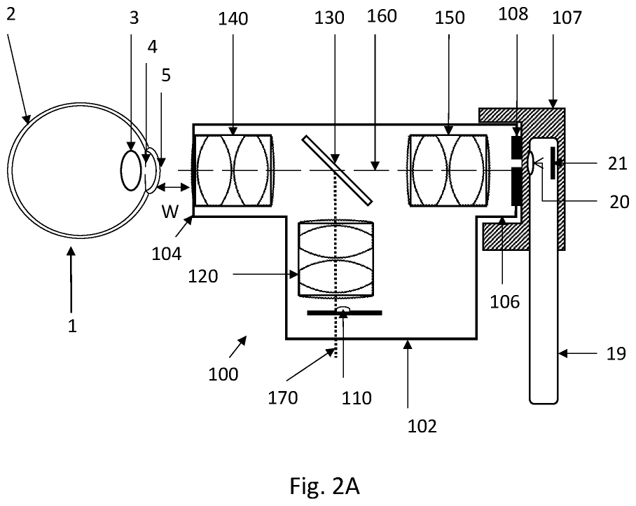

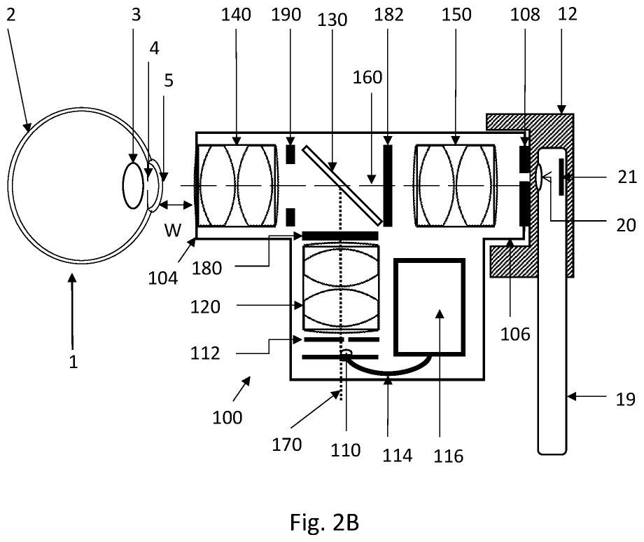

FIG. 2A, shows an example of an adaptor 100 according to the present disclosure together with an eye 1 and an image capturing device 19 for capturing an image of the retina of the eye. The adaptor uses a beam splitter 130 to separate light on the imaging and illumination paths.

The eye 1 comprises a cornea 5, a pupil 4, a crystalline lens 3 and the retina 2 at the back of the eye. The pupil 4 is an aperture surrounded by an iris of the eye and lies in a pupil plane extending in the direction from the top to the bottom of FIG. 2A.

The image capturing device 19 is a portable device including a camera, such as but not limited to, a smart phone, a digital camera, a tablet computer, a drone etc. The camera includes a lens 20 and a detector 21 such as a charge coupled device (CCD) sensor. The camera captures images by focusing optical radiation through the lens 20 to form an image on the detector 21.

The adaptor 100 is configured for attachment to the portable image capturing device 19 and to direct optical radiation on an illumination path into the eye 1 and on an imaging path reflected back from the retina of the eye through the adaptor 100 to the camera of the image capturing device 19. The adaptor 100 may comprise a housing 102 including a distal end 104 which is to be positioned a working distance W away from the eye and a proximal end 106 which is to be attached to the portable image capturing device.

The adaptor 100 comprises an illumination unit 110, a condensing lens system 120; a beam splitter 130; an objective lens system 140 and a secondary lens system 150. The objective lens system 140 and the secondary lens system 150 share a first optical axis 160 shown in dashed lines in FIG. 2A. The condensing lens system 120 has a second optical axis 170 shown in dotted lines, which is at an angle to the first optical axis 160. In the example of FIG. 2A, the second optical axis 170 is orthogonal to the first optical axis 160, but in other examples the second optical axis may be at an angle of less than 90 degrees to the first optical axis. The condensing lens system 120 and illumination unit 110 may be positioned to one side of, above, or below, the objective lens system 140 and the secondary lens system 150.

The beam splitter 130 is positioned between the objective lens system 140 and the secondary lens system 150 and may, for example, be positioned on the first optical axis 160. The condensing lens system 120 may comprise one or more lenses and is positioned between the illumination unit 110 and the beam splitter 130.

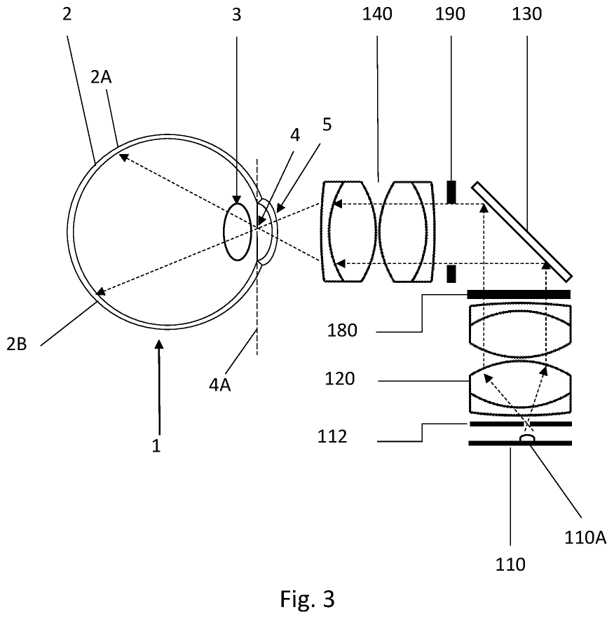

The illumination unit 110 is configured to direct optical radiation from the illumination unit through the condensing lens system 120 to the beam splitter 130. The beam splitter 130 is configured to direct the optical radiation from the condensing lens system to the objective lens system 140 for illuminating the retina 2. This path of optical radiation from the illumination unit 110, through the condensing lens system 120 and reflected by the beam splitter 130 through the objective lens 140 system to the eye 1 may be referred to as the illumination path. An example of the illumination path is shown in dashed lines in FIG. 3.

The objective lens system 140 may comprise one or more lenses and is configured to focus the optical radiation on the illumination path onto a focal plane positioned between the cornea 5 and the backside of the crystalline lens 3 of the eye. The back, or backside, of the crystalline lens 3 is the side of the crystalline lens which is nearest to the retina 2 and furthest from the cornea 5. In the example of FIG. 3, the objective lens system 140 is configured to focus light on the illumination path onto the pupil plane of the eye, so in the example of FIG. 3, the focal plane is the pupil plane of the eye. Thus, when the adaptor 100 is held at the working distance W away from the eye 1, the objective lens system 140 and the cornea 5 may focus light on the illumination path onto the pupil plane of the eye. In other examples, the objective lens system may be configured to focus light on the illumination path to a focal plane which is parallel to the pupil plane, and positioned between the pupil plane and the cornea, or between the pupil plane and the back of the crystalline lens 3.

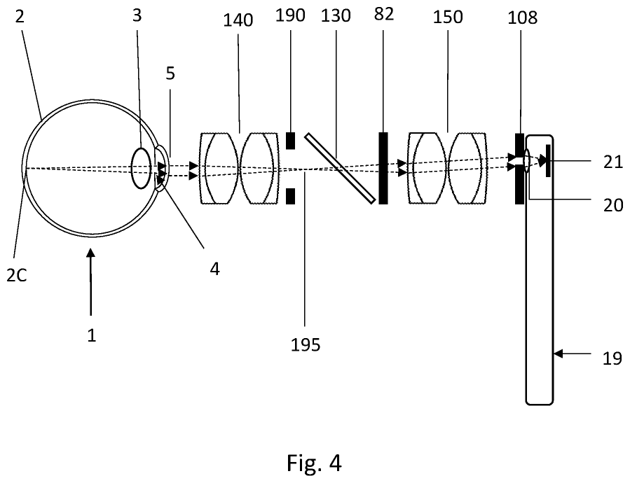

The objective lens system 140 is further configured to direct optical radiation reflected back from the retina on an imaging path to the secondary lens system 150. An example of the imaging path is shown by the dashed lines in FIG. 4. The secondary lens system 150 comprises one or more lenses and is configured to direct the optical radiation on the imaging path into the camera of the image capturing device 19. For example, the imaging path optical radiation may be directed through the lens 20 of the camera such that the optical radiation can be focused onto the detector 21 of the camera.

In order to reduce or prevent light on the illumination path from interfering with light on the imaging path, the system of lenses in the adaptor has at least one of the following offsets:
a) the illumination unit 110 may be offset from the second optical axis 170 of the condensing lens system 120; and/or
b) the adaptor may have an output aperture 108 and a centre of the output aperture 108 of the adaptor 100 may be offset from the first optical axis 160 of the adaptor. In this way when, in use, a camera is posited adjacent the output aperture, the camera will be offset from the first optical axis 160.

In some implementations, the illumination unit 110 may be offset from the second optical axis 170, while the centre of an output aperture 108 of the adaptor 100 is not offset from the first optical axis 160. In other examples, the centre of an output aperture 108 of the adaptor 100 is offset from the first optical axis 160, while illumination unit 110 may be offset from the second optical axis 170. In still other implementations, both the illumination unit 110 is offset from the second optical axis 170 and the centre of an output aperture 108 of the adaptor 100 is offset from the first optical axis 160. The offset of the centre of the output aperture from the first axis may be orthogonal to the offset of the illumination unit from the second optical axis.

By providing on or both of these offsets, overlap of the illumination path optical radiation and the imaging path optical radiation at the focal plane in the eye may be reduced or avoided. In this way interference between the imaging path and the illumination path may be reduced or minimised and image quality improved. Furthermore, the inventor has found that this arrangement can separate or reduce overlap of the imaging and illumination paths at the focal plane in the eye, even when eye is in a non-mydiatric condition (i.e. when the pupil is not dilated). Therefore the adaptor of the current disclosure may be used for non-mydiatric fundus photography. Non-mydiatric fundus photography is safer as it avoids the administration of medicine and may be carried out in a wider range of settings.

Offsetting both the illumination unit from the second optical axis and the output aperture from the first optical axis has a synergistic effect, as the offsets may move the illumination path and imaging path in opposite directions on the focal plane. This approach of offsetting both the illumination unit and the output aperture may significantly reduce overlap without unduly distorting the image, as each offset may be relatively small, but the focal points of the imaging and illumination radiation on the focal plane are moved apart by a total of both the offsets.

The offset, or each of the offsets where there are two offsets, may be equivalent to half the diameter of the pupil of an un-dilated eye. For example, the offset, or each one of the offsets, may be between 1 mm and 1.5 mm. In some examples the offset, or offsets are fixed. In other examples, the offset, or at least one of the offsets, is adjustable.

FIG. 2B shows an example of an adaptor according to the present disclosure, which is similar to that shown in FIG. 2A, but which includes additional components. Like parts have like reference numerals and functionality as in FIG. 2A and will not be described further below. The additional components are an illumination aperture 112, illumination unit circuitry 114, battery 116, polarizers 180, 182 and an imaging aperture stop 190. Any one, or a combination or some, or all, of these additional components may be added to the adaptor and are described briefly below.

The illumination unit 110 comprises a source of optical radiation 110A, similar to the illumination unit of FIG. 2A. The illumination unit 110 may further comprise an illumination aperture 112 as shown in FIG. 2B. In the arrangement of FIG. 2B, light travels from the source of optical radiation 110A through the illumination aperture 112 to the condensing lens system 120. The illumination aperture 112 may be formed in a housing of the illumination unit 110 or may be formed by a separate aperture stop member, such as a plate, which is separate from the housing of the illumination unit. The source of optical radiation 110A may be a point source, which approximates light emitted from a single point, such as a single LED. The use of an illumination aperture 112 may allow a wider choice of sources of optical radiation while still approximating a point source.

Where the illumination unit 110 does not have an illumination aperture, the offset of the illumination unit 110 from the second optical axis 170 may be achieved by offsetting the source of optical radiation 110A from the second optical axis 170. Where illumination unit 110 has an illumination aperture 112, the offset of the illumination unit 110 from the second optical axis 170 may be achieved by offsetting the illumination aperture 112 from the second optical axis 170.

The adaptor 100 may further comprise a battery 116 to power the illumination unit 110 and electrical circuitry 114 for passing the electrical power to the illumination unit and/or controlling the illumination unit. In other implementations, the illumination unit may be powered by an external power source.

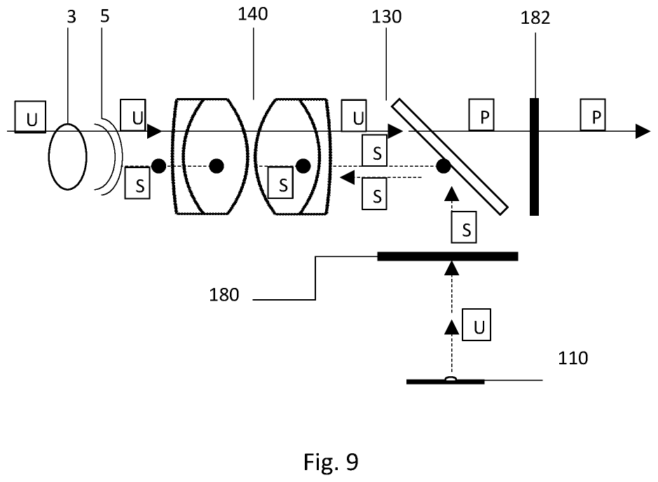

A first polarizer 180 may be positioned between the illumination unit 110 and the beam splitter 130 and a second polarizer 182 may be positioned between the objective lens system 140 and the secondary lens system 150. In the example of FIG. 2B, the second polarizer 182 is positioned between the beam splitter 130 and the secondary lens system 150. The first polarizer 180 and the second polarizer 182 have different polarizations. The polarizers 180, 182 are to filter out or reduce reflections from the cornea 5, objective lens system 140 and/or secondary lens systems 150, which might otherwise interfere with the illumination and/or imaging paths, as is discussed in more detail below in the description of FIG. 9.

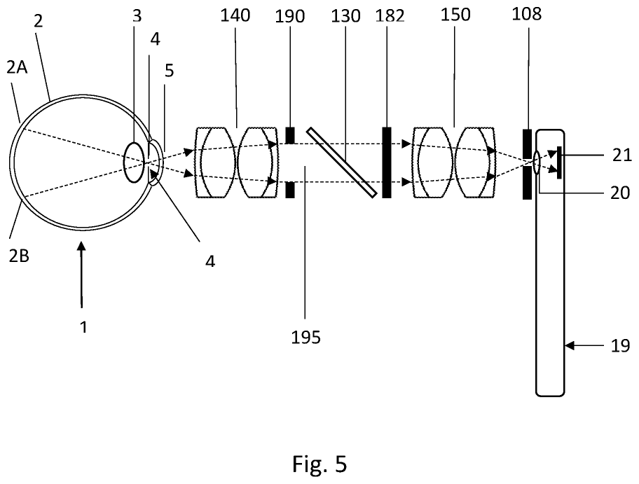

An imaging aperture stop 190 may positioned on the first optical axis 160 between the objective lens system 140 and the beam splitter 130. This limits the optical radiation on the imaging path back to the secondary lens system 150 and is discussed in more detail below in the description of FIGS. 4 and 5.

FIG. 3 shows an example of the illumination path in an adaptor according to the present disclosure. For simplicity, only the components of the adaptor on the illumination path are shown. The source of optical radiation 110A of the illumination unit 110 generates optical radiation which exits the illumination unit 110 through illumination aperture 112 and enters the condensing lens system 120. The light exiting the illumination aperture 112 approximates a point source and two divergent rays of light from this point source are shown in dashed lines in FIG. 3. The light from the illumination unit 110 is converged by the condensing lens system 120 into near collimated, i.e. substantially parallel, rays of light that are incident on the beam splitter 130. The beam splitter 130 reflects at least a portion of these light rays towards the objective lens system 140 and the objective lens system 140 directs the light rays towards the eye 1.

The light passes through the cornea 5, pupil 4 and crystalline lens 3 to illuminate the retina of the eye 2. The objective lens system 140 focuses the light on the illumination path onto a focal plane which is positioned between the cornea 5 and a backside of the crystalline lens 3 of the eye 1. In the example of FIG. 3, the focal plane is the pupil plane, which is shown by the dashed line 4A in FIG. 3 and extends through the pupil 4. In other examples the focal plane may be another plane parallel to the pupil plane, but extending through a different location between the cornea 5 or the back of the crystalline lens 3. After passing through the focal plane, where the light rays of the illumination path may be focused onto a small spot, the light rays diverge and reach different areas of the retina 2 at the back of the eye.

The two light rays shown in FIG. 3 may be the two outermost light rays of the illumination path and thus may define the extent of illumination of the retina 2 between points 2A and 2B. The outermost light rays may be defined by the optical components of the adaptor including the condensing lens, beam splitter and objective lens. In FIG. 3, the imaging field aperture stop 190 limits the extent of the illumination by blocking lights rays which are outside the relatively wide aperture of the imaging field aperture stop 190. The angle through which the retina is illuminated is known as the field of view. In examples of the present disclosure, the field of view of the retina may be at least 30 degrees measured from the centre of the pupil of the eye 1. The width of the aperture 190 depends on the focal length of the objective lens and the desired field of view. In some examples, the width of the aperture may be chosen to provide a field of view of between 30 to 50 degrees on the retina. The illumination path light rays are reflected by the retina and some of the reflected rays pass back through the pupil 4 on an imaging path which continues back through the adaptor to an image capturing device, as will be described below with reference to FIGS. 4 and 5.

FIG. 4 shows an example of the imaging path in an adaptor according to the present disclosure. Like parts have like reference numerals as in FIGS. 2A and 2B. For simplicity, only the components of the adaptor on the imaging path are shown. Light is reflected from various points on the retina 2 back though the crystalline lens 3 and the cornea 5 into the adaptor. The crystalline lens 3 and the cornea 5 may refract the reflected light to direct the reflected light back into the objective lens system 140 of the adaptor. FIG. 4 uses dashed lines to illustrate the light rays of light reflected from a particular point 2C on the retina.

The objective lens system 140 is configured to form a real intermediate image 195 of the retina, at a location between the objective lens system 140 and the secondary lens system 150, with the optical radiation reflected from the retina. The objective lens system 140 may be configured to form the real intermediate image 195 between the objective lens system 140 and the secondary lens system 150. For instance, the real intermediate image 195 may be formed between the objective lens system 140 and the beam splitter 130 or between the beam splitter 130 and the secondary lens system 150. In the example of FIG. 4, the objective lens system 140 is configured to form the real intermediate image 195 between the imaging field aperture stop 190 and the beam splitter 130. The exact location of the real intermediate image 195 may vary slightly depending on any refractive errors of the subject's eye 1.

After the intermediate image 195, light on the imaging path diverges again until reaching secondary lens system 150. Some or all of the light on the imaging path is transmitted by the beam splitter 130 through the polariser 82 (if present) and into the secondary lens system 150. The secondary lens system 150 may be configured to output parallel light rays which are focusable by the camera of the portable device to form an image of the retina. Thus the secondary lens system may collimate the lens and pass the collimated light through the output aperture 108 of the adaptor. Thus, in the use the imaging path light is directed into the lens 20 of the camera of the portable device 19 which is attached to the proximal end of the adaptor. The camera lens 20 converges the light rays onto the camera detector 21. The image formed on the detector 21 may be recorded and processed by the hardware and/or software of the portable image capturing device 19.

FIG. 5 is another diagram illustrating the imaging path in an adaptor according to the present disclosure. For simplicity, only the components of the adaptor on the illumination path are shown and like parts have like reference numerals as in FIG. 4. While FIG. 4 shows the light rays reflected from a single point 2C near the centre of the retina 2, FIG. 5 is a field of view analysis which shows chief rays reflected from two of the outermost points 2A, 2B of the field of view on the retina. The field of view may be limited by the aperture stop 190 as the aperture stop may block light rays which do not pass through the relatively wide aperture of the aperture stop.

The reflected light rays are shown by dashed lines in FIG. 5. The reflected rays converge at the focal plane where they may be focused into a small spot. In the example of FIG. 5, the focal plane is the pupil plane 4A, but in other examples the focal plane may be another plane parallel to the pupil plane, but extending through a different location between the cornea 5 or the back of the crystalline lens 3. After passing through the focal plane, the light rays diverge and are refracted by the objective lens system 140 which directs the light rays on the imaging path towards the secondary lens system 150. The reflected light rays pass from the objective lens system 140 through the imaging field aperture stop 190. The imaging field aperture stop 190 determines the field of view of the optical system. The collimated rays may be converged again by the secondary lens system towards the output aperture 108. After passing through the output aperture the chief light rays are refracted by the lens 20 of the camera of the portable image capturing device and reach different positions on the camera detector 21.

It will be appreciated that FIG. 4 shows light rays reflected from a central part of the retina onto the imaging pathway, while FIG. 5 shows light rays reflected from outer parts of the field of view of the retina onto the imaging pathway. Thus the real intermediate image 195 may comprise the light rays focused at the centre of the real intermediate image as shown in FIG. 4 and light rays focused at edges of the real intermediate image as shown in FIG. 5. That is, as the real intermediate image 195 is not a point, the light rays on the illumination path at the real intermediate image are focused into an area rather than a single point. The size of the real intermediate image may in some examples be approximately the same as the size of the aperture of the imaging aperture stop 190.

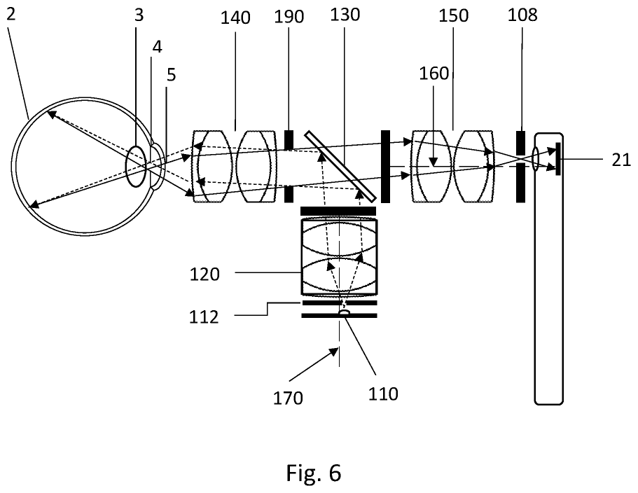

FIGS. 6 to 8 show both the illumination pathway and the imaging pathway, in an example of an adaptor according to the present disclosure. The illumination pathway is shown by dotted lines, while the imaging pathway is shown by solid lines. Like parts are denoted by like reference numerals as in FIGS. 2A, 2B and 3-5.

The illumination and imaging pathways travel a similar route between the beam splitter 130 and the retina 2, but the angles of travel are different due to the output aperture 108 being offset from the first optical axis 160 of the secondary lens system and/or due to the illumination unit 110 being offset from the second optical axis 170 of the condensing lens system 120 (e.g. by the illumination aperture 112 being offset from the second optical axis 170).

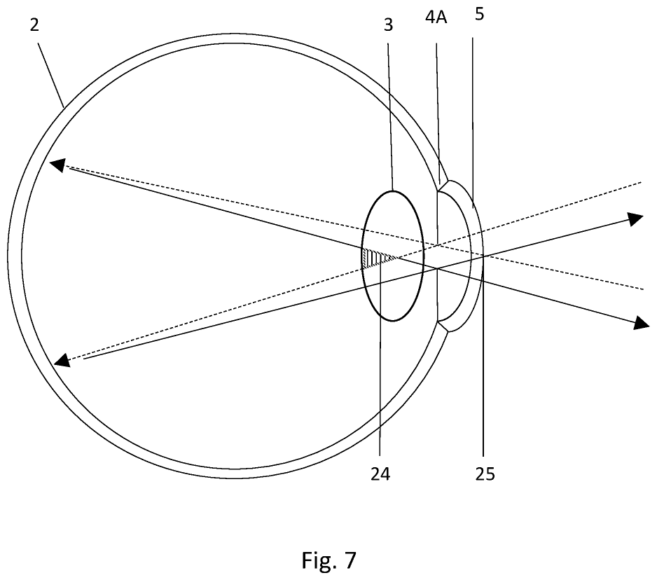

The illumination pathway and imaging pathway both converge onto the same focal plane within the eye. The focal plane may be any plane between the cornea 5 and the back of the crystalline lens, but in the example of FIGS. 6 to 8, the focal plane is the pupil plane of the eye. As can be seen in FIG. 6 and FIG. 7, the illumination path and the imaging path converge onto different parts of the focal plane. In this way overlap of the imaging pathway and illumination pathway in the cornea 5 and crystalline lens 3 is reduced or minimized.

The objective lens system 140, condensing lens system 120, beam splitter 130 and the secondary lens system 150 are configured such that non-overlapping real images of the illumination aperture 112 of the illumination unit and the output aperture 108 of the adaptor are formable on the focal plane. This can be seen by the ray tracing in FIGS. 6 and 7, which shows the illumination path light rays and imaging path light rays converged to different points on the same focal plane, which is the pupil plane in this example. Thus real images of the illumination aperture and the output aperture may be formed at the same axial location, in the axial direction of the first optical axis, but at different transverse locations relative the first optical axis.

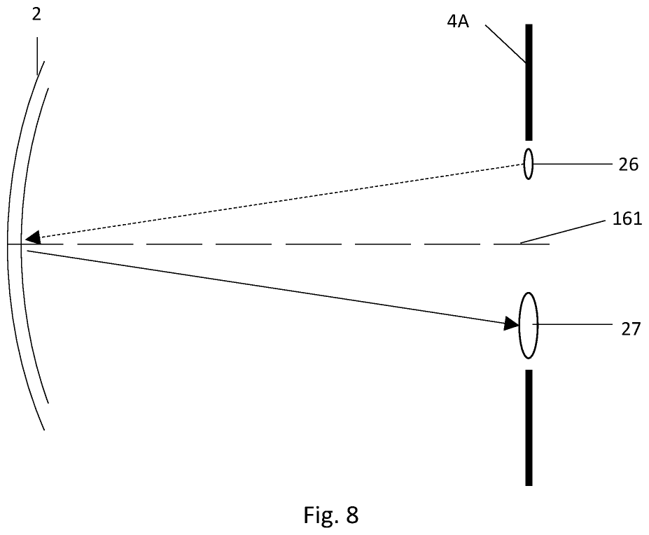

FIG. 8, shows an example of real images of the illumination aperture 26 and the output aperture 27 formed on the pupil plane 4A near opposite edges of the pupil 4. These real images may be configured in a symmetrical manner along the principal axis 161 of the eyeball (long dotted line), which may be aligned with the first optical axis 160 of the adaptor. This provides the best image of details of the retina 2 due to the combined effects of diffuse reflection in all directions and specular reflection along the opposite direction of incident angle (which gives rise to most surface details) from the retina 2.

FIG. 7 is a close up view of an example of the illumination pathway and imaging pathway light rays in the eye 1. In FIG. 7, dotted lines are used to represent the illumination pathway and solid lines to represent the imaging pathway and like reference numerals denote like parts as in FIG. 6. In the example of FIG. 7, the light rays do not overlap in the pupil plane 4A, as the illumination pathway and imaging pathway are converged to opposite sides of the pupil. However, there is a limited overlap 24 between the imaging and illumination pathways in the crystalline lens 3. There is also a limited overlap 25 between the imaging and illumination pathways in the cornea 5. As the light rays in FIG. 7 show the outermost light rays of the field of view, FIG. 7 shows the extent of overlap between the illumination and imaging pathways in the eye.

The crystalline lens 3 and cornea 5 of an eye are optically dense material that will reflect and scatter light radiation on the illumination pathway (dotted lines) in all directions. Therefore, when the illumination pathway overlaps with the imaging pathway (solid lines) in the eye, the scattered light from the illumination pathway will cause degradation of the final image, captured by the camera at the end of the adaptor. Accordingly, as described above, the adaptor of the present disclosure may be configured to converge the illumination pathway and imaging pathway to different parts of the focal plane (e.g. the pupil plane 4A). For instance, the pathways may be converged to small spots near opposite edges of the pupil. This configuration reduces or minimizes the overlapping area of the imaging and illumination pathways in crystalline lens (24) and cornea (25), thereby improving image quality.

In examples of the present disclosure, the offset of the centre of the output aperture 108 from the first optical axis 160 may be between 1 and 1.5 mm, which corresponds to half a diameter of the pupil of an average human eye which is being illuminated. Likewise, the offset of the illumination unit 110 from the second optical axis 170 is between 1 and 1.5 mm. The degree of separation of the illumination and imaging pathways in the pupil plane is related to the size of the offset of the output aperture from the first optical axis (‘the first offset’) and the size offset of the illumination unit from the second optical axis (‘the second offset’). Thus larger offsets may cause a larger separation of the illumination and imaging paths in the pupil plane.

The first offset may deviate the intersection of the imaging pathway with the pupil plane away from the centre of the pupil, while the second offset may deviate the intersection of the illumination pathway with the pupil plane away from the centre of the pupil. The exact relationship between the offset and the deviation depends upon the focal lengths of the objective lens system and secondary lens system, but in general the offset and the deviation may be of a similar order of magnitude. If the ratio of the focal length of the objective lens system to the focal length of the secondary lens system is 1, then the magnitude of the first (or second) offset is equal to the magnitude of the deviation of the imaging (or illumination) pathway from the centre of the pupil. The direction of the first and second offsets may be chosen such that a direction in which the first offset deviates the imaging pathway away from the centre of the pupil is opposite to the direction in which the second offset deviates the illumination pathway from the centre of the pupil, e.g. as shown in FIGS. 6 to 8 where the imaging pathway is deviated downwards in the pupil plane and the illumination pathway is deviated upwards in the pupil plane; in this way the first offset and second offset may act in a cumulative fashion to increase the separation of the imaging and illumination pathways in the pupil plane.

It is to be understood that the imaging and illumination pathways shown in FIGS. 6 to 8 are merely by way of example and that other pathways are possible within the scope of the present disclosure. For example, the real intermediate images 26, 27 in the eye in FIG. 8 are offset from the first optical axis of the objective lens system (assumed to be aligned with principle axis of the eye 161), in the vertical direction on the focal plane. However, in other examples the real intermediate images 26, 27 may be offset in a different direction on the focal plane. For instance, the real intermediate images 26, 27 may be offset to the left or right of the first optical axis. Further, while the real intermediate images 26, 27 are located near the edges of the pupil 4 in FIG. 8, in other examples the real intermediate images may be further away from edges of the pupil, but still offset from the first optical axis (and principal axis of the eye 161). Further, while the illumination unit real image 26 is shown at the top of the pupil and the output aperture real image 27 is shown at the bottom of the pupil in FIG. 8, in other examples the adaptor may be configured such that their positions are reversed and the illumination unit real image 26 is at the bottom of the pupil and the output aperture real image 27 is at the top of the pupil.

In still other examples, there may be a degree of overlap between the real intermediate image of the illumination unit 26 and the real intermediate image 27 of the output aperture in the focal plane, especially if the images are larger than shown in FIG. 8, but with the centre of the two images offset from each other so as to reduce the overlap. However, the quality of the final image captured by the camera is further improved if there is no overlap on the focal plane.

While in FIG. 8 both the illumination aperture real image 26 and the output aperture real image 27 are offset from the first optical axis (and principle axis 161 of the eye) at the focal plane. However, in other examples just one of these real images may be offset. For instance, if the output aperture 108 is offset from the first optical axis 160, but the illumination unit 110 is not offset from the second optical axis 170, then the real image 26 of the illumination unit on the focal plane may be centred on the first optical axis, while the real image 27 of the output aperture may be offset from the first optical axis. On the other hand, if the illumination unit 110 is offset from the second optical axis 170, but the output aperture 108 is not offset from the first optical axis 160, then the real image 26 of the illumination unit on the focal plane may be centred on the first optical axis, while the real image 27 of the output aperture on the focal plane may be offset from the first optical axis. However, where both the output aperture is offset from the first optical axis and the illumination unit is offset from the second optical axis, then both the real images 26, 27 on the focal plane are offset from the first optical axis and this provides a synergistic effect as the separation of the real images in the focal plane is greater and thus there is greater separation of the illumination pathway and imaging pathway in the eye reducing interference due to scattered optical radiation.

FIG. 9 illustrates the effect of the polarizers 180, 182 on the light rays of the imaging and illumination pathways in an adaptor according to an example of the present disclosure. Like parts are denoted by like reference numerals as in the previous figures. The illumination pathway is represented by dashed lines, while the imaging pathway is represented by solid lines. For simplicity, only parts related to the polarization are included in FIG. 9.

The adaptor may comprise a first polarizer 180 positioned between the illumination unit 110 and the beam splitter 130 and a second polarizer 182 positioned between the objective lens system 140 and the beam splitter 130, wherein the first polarizer 180 and the second polarizer 182 have different polarizations. The beam splitter may be a polarizing beam splitter which is configured to transmit or reflect incident light depending upon the polarization of the incident light.

For example, the beam splitter may comprise a wire grid for splitting an incident beam according to polarization. In one example, the first polarizer may be configured to transmit light having a first polarization, such as S-polarization, and block light having a second polarization, such as P-polarization, while the second polarizer may be configured to reflect light having the first polarization and transmit light having the second polarization. While S and P polarization are used as examples below, it is to be understood that in other implementations different types of polarization may be used.

FIG. 9 shows an example according to the present disclosure, in which light is emitted from the illumination unit 110 as unpolarized light (labelled “U” in FIG. 9). The unpolarised light passes through first polarizer 180, which acts as a pre-polarizing filter, and thus becomes polarized light. For example the light may become S-polarized light (labelled “S” in FIG. 9). The polarizing beam splitter 130 may reflected the S-polarized light by 90 degrees as S-polarized light in the direction towards the objective lens system 140.

Some of the S-polarized light on the illumination path may be reflected back by the smooth surfaces of the objective lens system 140, cornea 5 and/or crystalline lens 3. However, due to the smooth surfaces, such reflected light maintains the S-polarization. In FIG. 9, this reflected light with the S-polarization is shown by the dashed lines with a circle, instead of an arrow, at the end. As shown in FIG. 9, the reflected S-polarized light may be blocked by the wire-grid polarizing beam splitter 130 which may be configured to reflect and not transmit S-polarized light.

In contrast, light reflected by the retina 2 does not maintain the polarization and thus becomes unpolarised light (labelled “U” in FIG. 9), due to the rough nature of the retina. Therefore at least a portion of the light reflected from the retina on the imaging pathway may be transmitted through the beam splitter 130. For example, the beam splitter 130 may be configured to transmit P-polarized light, such that after passing through the beam splitter the imaging pathway light becomes P-polarized light. After passing through the beam splitter 130 the imaging pathway light travels to the second polarizer 182. The second polarizer 182 acts as a post-polarizing filter (9) and may remove unwanted reflections from the smooth surfaces of the optical components, such as the lenses or the beam splitter. The light passing through the second polarizer 182 is thus P-polarized light (labelled “P” in FIG. 9) and thus contains information mostly relating to the retina, as reflections from other sources have been filtered out. This is because the second polarizer 182 filters out light with an S-polarization and so light from the imaging pathway and any light reflected by the various lenses is filtered out and does not reach the camera of the imaging device.

In some examples, the beam splitter may not be a polarizing beam splitter, but a similar effect may be achieved by the combination of the first polarizer and the second polarizer. In other examples a similar effect may be achieved by using a polarizing beam splitter and omitting the first polarizer and the second polarizer. However, by combining both a polarizing beam splitter and a pair of polarizers, a better filtering effect may be achieved.

In some examples, where the beam splitter 30 is a polarizing beam splitter, the beam splitter may be configured such that maximum reflection occurs when optical radiation is incident at an angle of 45 degrees to a plane of the beam splitter and such that the extinction ratio of unpolarized to polarized optical radiation is at least 1:100 when optical radiation is incident at an angle of between 25 degrees and 65 degrees to the plane of the beam splitter. This configuration makes it possible to filter out unwanted reflections over a relatively wide field of view.

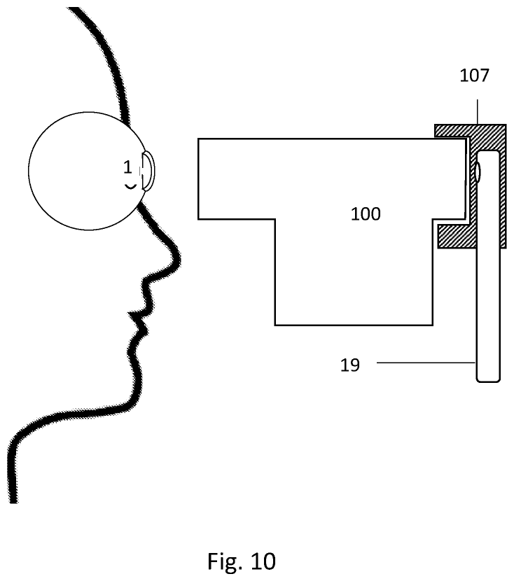

FIG. 10 shows an example of an adaptor 100 according to the present disclosure when in use. A proximal end 106 of the adaptor 100 is attached to a portable image capturing device 19 and a distal end 104 of the adaptor is positioned adjacent an eye 1 which is to be photographed. The adaptor 100 may be held a working distance W away from the eye. As the adaptor and the lenses of the adaptor are not in physical contact with the subject's eye 1, the procedure is relatively safe and does not require any special materials for protecting the eye. A suitable working distance W may be determined by a user of the adaptor, such as a person using the image capturing device to take a photograph of the retina of the eye.

The subject 10 whose eye 1 is to be photographed may be different to the person taking the photograph. For instance, the user may stand opposite the subject and hold the adaptor 100 and image capturing device and move the distal end of the adaptor in proximity to the subject's eye 1. The user may determine from a display of the portable image capturing device 19 when the retina is in focus. For instance, hardware and/or software of the portable image capturing device may indicate when light from the illumination unit is focused on the desired focal plane, e.g. pupil plane, of the user's eye. The portable image capturing device 19 may have a software application installed thereon for guiding the user to capture an image of the subject's retina.

In the example of FIG. 10, the adaptor 100 comprises a mount 107 which is separable from the adaptor housing. The mount 107 is attachable to the adaptor housing 100 and the portable image capturing device 19. For example, the mount 107 may include an image capturing device receiving portion, such as a slot in which to receive the image capturing device, and an adaptor receiving portion to receive the adaptor housing. In other examples, the mount 107 may be integral with the adaptor housing. It is to be appreciated that the mount 107 shown in FIG. 10 is just one example, and other types and shapes of mount, and/or other methods of attaching the adaptor to the portable image capturing device, are possible and within the scope of the present disclosure.

The adaptor 100 may be attached to the portable image capturing device 19 in a position such that optical radiation exiting an output aperture of the adaptor enters a camera of the image capturing device.

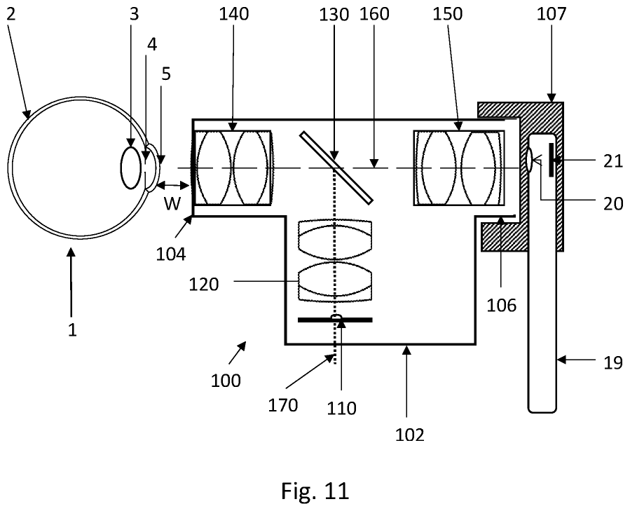

FIG. 11 shows a further example of an adaptor according to the present disclosure. Like reference numerals denote like parts and have the same functionality as in the examples above. The adaptor of FIG. 11 may direct the light as discussed above in relation to FIGS. 3 to 10. However, the example adaptor of FIG. 11 differs from the adaptor of FIGS. 2A and 2B in that there is no output aperture stop. Rather, light exits the adaptor through the secondary lens system 150 and travels from the secondary lens system 150 to the camera lens 20 of the image capturing device 19, without passing through an output aperture stop. The principle of operation is, however, similar to that described in the description and figures above.

The adapter 100 is configured to attach to the portable image capturing device 19 in a position in which the camera of the portable image capturing device is offset from the first optical axis 160. The camera being offset form the first optical axis means that the centre of the camera lens 20 is offset from the first optical axis 160 of the secondary lens system 150. This has the same effect as offsetting an output aperture 108 of the adaptor from the first optical axis in FIGS. 2-10. In relation to FIG. 8, which refers to a real intermediate image 27 of the adaptor output aperture, in the case where there is no output aperture as in the adaptor of FIG. 11, the real intermediate image will be of the camera aperture, e.g. the camera lens 20.

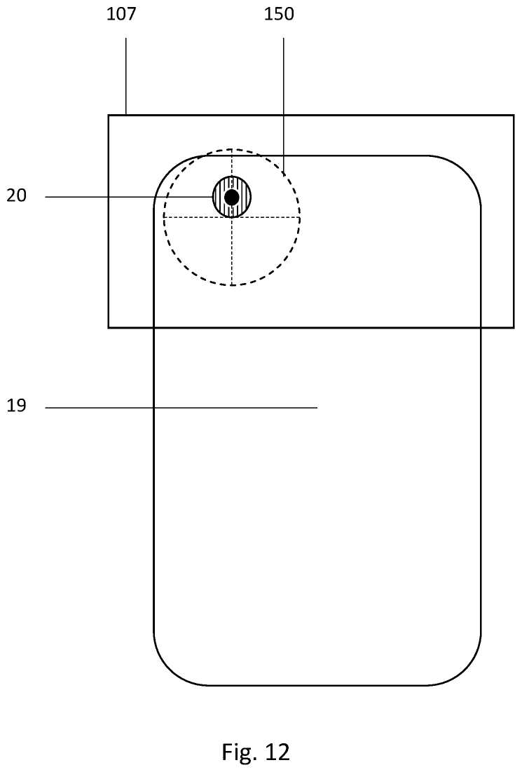

FIG. 12 is a schematic diagram showing an example of the relative positions of the mount 107, the image capturing device 19, camera lens 20 of the image capturing device and the secondary lens system 150 (shown in dashed lines with cross-hairs showing the centre through which the optical axis 160 passes). It will be appreciated that the camera lens 20 is offset from the optical axis 160 of the secondary lens system. The same relative positioning may be achieved by any of the apparatus described above including those of FIG. 2A, 2B or 11.

Referring again to FIG. 11, it will be appreciated that the adaptor 100 is an adaptor for attachment to a portable image capturing device 19 including a camera for capturing images of a retina of an eye and that the adaptor comprises an illumination unit 110, a beam splitter 130, an objective lens system 140 and a secondary lens system 150.

The illumination unit 110 comprises an optical radiation source 110A and the illumination unit 110 is configured to direct optical radiation from the optical radiation source 110A to the beam splitter 130. The beam splitter 130 is positioned between the objective lens system 140 and the secondary lens system 150. The beam splitter 130 is configured to direct the optical radiation to the objective lens system 140 for illuminating the retina 2 of the eye.

The objective lens system 140 is configured to focus the optical radiation on a focal plane positioned between a cornea 5 and a backside of a crystalline lens 3 of the eye and direct optical radiation reflected from the retina towards the secondary lens system 150. For example, the focal plane may be a pupil plane of the eye.

The secondary lens system 150 is configured to direct the reflected optical radiation to an exterior of the adaptor 100 for reception by a camera of a portable image capturing device 19 which is to be attached to the adaptor 100.

The objective lens system 140 and the secondary lens system 150 share a first optical axis 160. The adaptor 100 is configured to attach to the portable image capturing device 19 in a position in which the camera of the portable image capturing device is offset from the first optical axis 160.

The offset of the camera of the portable image capturing device from the first optical axis 160 may prevent overlap, at the focal plane, between optical radiation focused on the focal plane by the objective lens system and optical radiation reflected by the retina.

In the example of FIG. 11, the illumination unit 110 is not offset from the second optical axis 170 of the condensing lens system 120. However, due to the offset of the camera from the first optical axis, a degree of separation of the imaging pathway and illumination pathways may be achieved within the eye 1.

In other examples, the adaptor of FIG. 11 may be modified to offset the illumination unit 110 from the second optical axis 170 of the condensing lens system, in a similar manner as described for FIGS. 2A, 2B and 3 above. In such cases the adaptor comprises a condensing lens system 120 positioned between the illumination unit and the beam splitter, wherein the condensing lens system has a second optical axis which is at an angle to the first optical axis and is offset from an illumination aperture associated with the illumination unit.

In examples of the present disclosure, as discussed above in relation to FIGS. 1-12, the adaptor may be configured such that the field of view of the retina is at least 30 degrees measured from the centre of a pupil plane of the eye.

In examples of the present disclosure the objective lens system 140 may include one or more lenses. In some examples the objective lens system 150 may contain at least one achromatic doublet lens. Likewise the secondary lens system may include one or more lenses. In some examples, the secondary lens system 150 may contain at least one achromatic doublet lens. Further, the condensing lens system may include one or more lenses. In some examples the condensing lens system may contain at least one achromatic doublet lens in order to further improve the image quality. The use of at least one achromatic doublet lens in one, some, or all of the objective lens system, secondary lens system and condensing lens system may help to reduce chromatic aberration in the optical system of the adaptor.

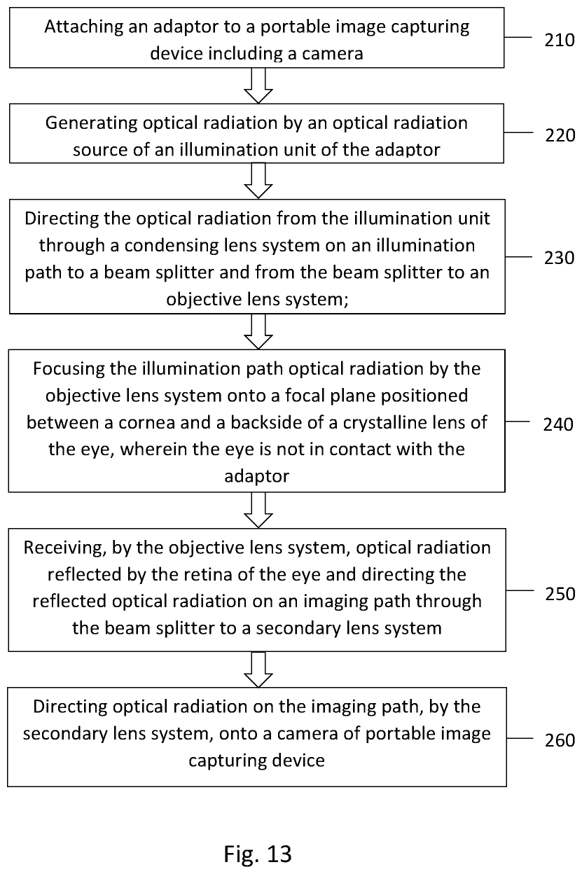

FIG. 13 is a method diagram showing an example method 200 of capturing an image of a retina of an eye. The method may use any of the adaptors described herein. The method comprises the following blocks:
Block 210: attaching an adaptor to a portable image capturing device including a camera;
Block 220: generating optical radiation by an optical radiation source of an illumination unit of the adaptor;
Block 230: directing the optical radiation from the illumination unit through a condensing lens system on an illumination path to a beam splitter and from the beam splitter to an objective lens system;
Block 240: focusing the illumination path optical radiation by the objective lens system onto a focal plane positioned between a cornea and a backside of a crystalline lens of the eye, wherein the eye is not in contact with the adaptor;
Block 250: receiving, by the objective lens system, optical radiation reflected by the retina of the eye and directing the reflected optical radiation on an imaging path through the beam splitter to a secondary lens system;
Block 260: directing optical radiation on the imaging path, by the secondary lens system, onto a camera of the portable image capturing device;

In the method of FIG. 13, the objective lens system and the secondary lens system share a first optical axis and the condensing lens system has a second optical axis which is at an angle to the first optical axis. The camera of the portable image capturing device is offset from the first optical axis and/or an illumination aperture associated with the illumination unit is offset from the second optical axis. Accordingly due to the offset(s) overlap of the imaging path and illumination path of the optical radiation in the eye is reduced, or eliminated, which leads to a higher quality image. Furthermore, as the illumination path and imaging path optical radiation may both be converged to relatively small spots on a focal plane in the eye, such as the pupil plane, it is possible to capture an image of the retina of the eye even when the eye is in an non-mydiatric state.

The above embodiments are described by way of example only. Many variations are possible without departing from the scope of the invention as defined in the appended claims.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

It is to be understood that any feature described in relation to any one example may be used alone, or in combination with other features described, and may also be used in combination with any features of any other of the examples, or any combination of any other of the examples.

1. An adaptor for attachment to a portable image capturing device for capturing images of a retina of an eye, the adaptor comprising:
an illumination unit;
a condensing lens system;
a beam splitter;
an objective lens system;
a secondary lens system;
an output aperture which is to be positioned adjacent a camera of the portable image capturing device;
the illumination unit being configured to direct the optical radiation on an illumination path from the illumination unit through the condensing lens system to the beam splitter;
the beam splitter being positioned between the objective lens system and the secondary lens system and being configured to direct the optical radiation on the illumination path to the objective lens system for illuminating the retina;
the objective lens system being configured to focus the optical radiation on the illumination path onto a focal plane positioned between a cornea and a backside of a crystalline lens of the eye and direct optical radiation reflected from the retina on an imaging path to the secondary lens system;
the secondary lens system being configured to direct the optical radiation on the imaging path through the output aperture;
wherein the objective lens system and the secondary lens system share a first optical axis;
wherein the condensing lens system has a second optical axis which is at an angle to the first optical axis;
and wherein at least one of:
the illumination unit is offset from the second optical axis and/or a centre of the output aperture of the adaptor is offset from the first optical axis.

2. The adaptor of claim 1 wherein an optical radiation source and/or an illumination aperture of the illumination unit is offset from the second optical axis.

3. The adaptor of claim 1 wherein the focal plane is a pupil plane of the eye.

4. The adaptor of claim 1, wherein the objective lens system, condensing lens system, beam splitter and the secondary lens system are configured such that non-overlapping real images of the illumination aperture of the illumination unit and the output aperture of the adaptor are formable on the focal plane.

5. The adaptor of claim 1, wherein the offset of the centre of the output aperture from the first optical axis is orthogonal to the offset of the illumination unit from the second optical axis.

6. The adaptor of claim 1, wherein the offset of the centre of the output aperture from the first optical axis is between 1 and 1.5 mm.

7. The adaptor of claim 1, wherein the offset of the illumination unit from the second optical axis is between 1 and 1.5 mm.

8. The adaptor of claim 1, wherein the objective lens system is configured to form a real intermediate image of the retina between the objective lens system and the secondary lens system with the optical radiation reflected from the retina.

9. The adaptor of claim 1, wherein the secondary lens system is configured to output parallel light rays which are focusable by the camera of the portable image capturing device to form an image of the retina.

10. The adaptor of claim 1, further comprising a first polarizer positioned between the illumination unit and the beam splitter and a second polarizer positioned between the objective lens system and the secondary lens system, wherein the first polarizer and the second polarizer have different polarizations.

11. The adaptor of claim 1, wherein the beam splitter is a polarizing beam splitter.

12. The adaptor of claim 1, further comprising an imaging field aperture stop between the objective lens system and the beam splitter.

13. The adaptor of claim 1 in combination with a portable image capturing device including a camera, wherein the adaptor is attached to the portable image capturing device in a position such that optical radiation exiting the output aperture of the adaptor enters the camera of the portable image capturing device.

14. The adaptor of claim 1, further comprising a housing including a distal end which is to be positioned a working distance away from the eye and a proximal end which is to be attached to the portable image capturing device.

15. The adaptor of claim 14 further comprising a mount which is separable from the housing, the mount being attachable to the housing and the portable image capturing device.

16. The adaptor of claim 1 wherein the offset, or at least one of the offsets, is adjustable.

17. An adaptor for attachment to a portable image capturing device including a camera for capturing images of a retina of an eye, the adaptor comprising:
an illumination unit;
a beam splitter;
an objective lens system;
a secondary lens system;
the illumination unit comprising an optical radiation source, the illumination unit being configured to direct optical radiation from the optical radiation source to the beam splitter;
the beam splitter being positioned between the objective lens system and the secondary lens system and being configured to direct the optical radiation to the objective lens system for illuminating the retina;
the objective lens system being configured to focus the optical radiation on a focal plane positioned between a cornea and a backside of a crystalline lens of the eye and direct optical radiation reflected from the retina towards the secondary lens system;
the secondary lens system being configured to direct the reflected optical radiation to an exterior of the adaptor for reception by a camera of a portable image capturing device which is to be attached to the adaptor;
wherein the objective lens system and the secondary lens system share a first optical axis;
and wherein the adaptor is configured to attach to the portable image capturing device in a position in which the camera of the portable image capturing device is offset from the first optical axis.

18. The adaptor of claim 17 wherein the offset of the camera of the portable image capturing device from the first optical axis prevents overlap, at the focal plane, between optical radiation focused on the focal plane by the objective lens system and optical radiation reflected by the retina.

19. The adaptor of claim 17, further comprising a condensing lens system positioned between the illumination unit and the beam splitter, wherein the condensing lens system has a second optical axis which is at an angle to the first optical axis and is offset from an illumination aperture associated with the illumination unit.

20. The adaptor of claim 17 wherein the field of view of the retina is at least 30 degrees measured from the centre of a pupil plane of the eye.

21. The adaptor of claim 1, wherein the objective lens system and the secondary lens system each contain at least one achromatic doublet lens.

22. The adaptor of claim 11 wherein the polarizing beam splitter is configured such that maximum reflection occurs when optical radiation is incident at an angle of 45 degrees to a plane of the polarizing beam splitter and when the extinction ratio of the unpolarized to polarized optical radiation is at least 1:100 when optical radiation is incident at an angle of 25 degrees and 65 degrees to the plane of the polarizing beam splitter.

23. A method of capturing an image of a retina of an eye comprising:
attaching an adaptor to a portable image capturing device including a camera;
generating optical radiation by an optical radiation source of an illumination unit of the adaptor;
directing the optical radiation from the illumination unit through a condensing lens system on an illumination path to a beam splitter and from the beam splitter to an objective lens system;
focusing the illumination path optical radiation by the objective lens system onto a focal plane positioned between a cornea and a backside of a crystalline lens of the eye, wherein the eye is not in contact with the adaptor;
receiving, by the objective lens system, optical radiation reflected by the retina of the eye and directing the reflected optical radiation on an imaging path through the beam splitter to a secondary lens system;
directing optical radiation on the imaging path, by the secondary lens system, onto a camera of portable image capturing device;
wherein the objective lens system and the secondary lens system share a first optical axis and the condensing lens system has a second optical axis which is at an angle to the first optical axis; and
wherein the camera of the portable image capturing device is offset from the first optical axis and/or an illumination aperture associated with the illumination unit is offset from the second optical axis.

24. The method of claim 23 wherein the eye is in a non-mydriatric state.
